(19) United States
(12) Patent Application Publication
(10) Pub. No.: US 2009/0099517 A1
(43) Pub. Date: Apr. 16, 2009

(54) REINFORCED, NON-COMPLIANT ANGIOPLASTY BALLOON

(75) Inventor: Christopher L. Steadham, Scottsdale, AZ (US)

Correspondence Address: C. R. Bard, Inc.;Bard Peripheral Vascular, Inc., 1415 W. 3rd St, PO Box 1740, Tempe, AZ, 85280-1740, US

(73) Assignee: C. R. Bard, Inc., Murray Hill, NJ (US)

(21) Appl. No.: 11/869,946

(22) Filed: Oct. 10, 2007

(51) Int. Cl. A61M 29/02

(52) U.S. Cl. 604/103.09

(57) ABSTRACT

An angioplasty balloon is disclosed and can include a body having a first end and a second end. An inflatable portion can be established between the first end and the second end. Moreover, a first reinforcing member can be wound around the body from the first end to the second end over the inflatable portion. Further, the first reinforcing member can be wound at a first winding angle with respect to a longitudinal axis. The first winding angle is in a range of fifteen degrees to forty-five degrees (15°-45°).

### FIELD OF THE DISCLOSURE

The present disclosure relates generally to surgical devices. More specifically, the present disclosure relates to angioplasty balloons.

### BACKGROUND

Vascular stenosis is an abnormal narrowing in a blood vessel. Vascular stenosis can include peripheral artery stenosis, coronary artery stenosis, carotid artery stenosis, and renal artery stenosis. There exist several ways to detect vascular stenosis. For example, a vascular stenosis can be detected using a stethoscope to amplify bruit, i.e., noise, within the blood vessel due to turbulent blood flow through the narrowed blood vessel. Alternatively, one or more imaging methods can be used to detect and locate a vascular stenosis. For example, ultrasound, magnetic resonance imaging, and computed tomography can be used to detect and locate a vascular stenosis.

A common cause of vascular stenosis is atherosclerosis. Atherosclerosis, aka, hardening of the arteries, is a disease that affects the arterial blood vessel. Atherosclerosis is caused by the formation of multiple plaques within the arteries. As plaque builds up within an artery, the diameter of the artery is reduced and results in a stenosis.

Vascular stenosis can be treated using an angioplasty balloon. An inflatable angioplasty balloon can be installed on a catheter and then, threaded through a patient's cardiovascular system to the stenosis. Once the angioplasty balloon is in place within the stenosis, the angioplasty balloon catheter can be inflated in order to expand the stenosis. Thereafter, the balloon catheter can be deflated and withdrawn from the patient. One risk associated with using an angioplasty balloon involves rupturing the balloon, e.g., due to over inflation.

Accordingly, there is a need for an improved angioplasty balloon.

### BRIEF DESCRIPTION OF THE DRAWINGS

FIG. 1 is a plan view of an angioplasty balloon delivery device;

FIG. 2 is a cross section view of an inflatable angioplasty balloon installed on an end of a delivery device;

FIG. 3 is a plan view of a first embodiment of an inflatable angioplasty balloon;

FIG. 4 is a plan view of a second embodiment of an inflatable angioplasty balloon; and

FIG. 5 is a flow chart illustrating an embodiment of a method of making an inflatable angioplasty balloon.

### DETAILED DESCRIPTION OF THE DRAWINGS

An angioplasty balloon is disclosed and can include a body having a first end and a second end. An inflatable portion can be established between the first end and the second end. Moreover, a first reinforcing member can be wound around the body from the first end to the second end over the inflatable portion. Further, the first reinforcing member can be wound at a first winding angle with respect to a longitudinal axis. The first winding angle is in a range of fifteen degrees to forty-five degrees (15°-45°).

In another embodiment, an angioplasty balloon is disclosed and can include a body having a first end and a second end. An inflatable portion can be established between the first end and the second end. A first reinforcing member can be wound around the body from the first end to the second end over the inflatable portion. Also, a second reinforcing member can be wound around the body from the second end to the first end over the first reinforcing member. The second reinforcing member can cross the first reinforcing member at a crossing angle. The crossing angle can be in a range of thirty degrees to ninety degrees (30°-90°).

In yet another embodiment, a method of making an angioplasty balloon is disclosed and can include inflating an angioplasty balloon and coating the angioplasty balloon, a first reinforcing member, or a combination thereof with an adhesive. Further, the method can include winding the first reinforcing member around the angioplasty balloon at a first winding angle with respect to a longitudinal axis of the angioplasty balloon. The first winding angle is in a range of fifteen degrees to forty-five degrees (15°-45°).

In still another embodiment, a method of making an angioplasty balloon is disclosed and can include inflating an angioplasty balloon and coating the angioplasty balloon, a first reinforcing member, a second reinforcing member, or a combination thereof with an adhesive. The method can also include winding the first reinforcing member around the angioplasty balloon and winding the second reinforcing member around the angioplasty balloon. The second reinforcing member can cross the first reinforcing member to form a crossing angle that is in a range of thirty degrees to ninety degrees (30°-90°).

### Description of an Angioplasty Balloon Delivery Device

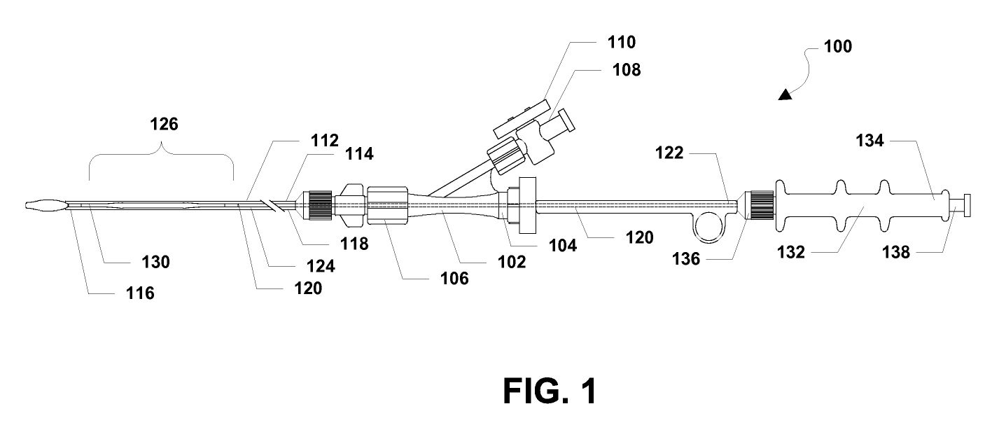

Referring to FIG. 1, an angioplasty balloon delivery device is shown and is generally designated 100. As shown, the angioplasty balloon delivery device 100 includes a body 102 having a proximal end 104 and a distal end 106. A first syringe attachment 108 can be formed in the body 102 between the proximal end 104 and the distal end 106. In a particular embodiment, the first syringe attachment 108 can be a Luer syringe attachment. The first syringe attachment 108 can provide fluid communication to a lumen formed within an outer sheath 112, described below. Further, the first syringe attachment 108 can include a stopcock 110.

FIG. 1 indicates that the angioplasty balloon delivery device 100 can include an outer sheath 112. The outer sheath 112 can include a proximal end 114 and a distal end 116. Further, the outer sheath 112 can extend from the distal end 106 of the body 102 of the angioplasty balloon delivery device 100. In particular, the proximal end 114 of the outer sheath 112 can be attached to the distal end 106 of the body 102 of the angioplasty balloon delivery device 100. The distal end 116 of the outer sheath 112 can be relatively soft and rounded. The outer sheath 112 can include a lumen 118 formed therein.

As illustrated in FIG. 1, the angioplasty balloon delivery device 100 can further include an inner carrier catheter 120. The inner carrier catheter 120 can extend through the body 102 of the angioplasty balloon delivery device 100 and into the lumen 118 formed in the outer sheath 112. The inner carrier catheter 120 can be coaxial with the outer sheath 112. Further, the inner carrier catheter 120 can include a proximal end 122 and a distal end 124. The inner carrier catheter 120 can be formed with a lumen (not shown) that can be sized to fit over a guide wire. In particular, the lumen of the inner carrier catheter 120 can fit over a 0.035 inch guide wire.

As shown in FIG. 1, an angioplasty balloon 126 can be compressed between the inner catheter 120, e.g., the distal end of the inner catheter 120, and the outer sheath 112. The distal end of the inner catheter 120 can include a first radiopaque band 128 and a second radiopaque band 130 positioned within the ends of the angioplasty balloon 126. With the aid of fluoroscopy, the radiopaque bands 128, 130 can facilitate positioning of the angioplasty balloon 126 within a patient.

A handle 132 can be attached to, or otherwise extend from, the proximal end 122 of the inner carrier catheter 120. The handle 132 can include a proximal end 134 and a distal end 136. The proximal end 134 of the handle 132 can include a second syringe attachment 138. In a particular embodiment, the second syringe attachment 138 can be a Luer syringe attachment. The second syringe attachment 138 can provide fluid communication with the lumen formed within the inner carrier catheter 120.

The angioplasty balloon delivery device 100 can also include a safety clip 140 installed between the body 102 of the angioplasty balloon delivery device 100 and the handle 128 of the inner carrier catheter 120. The safety clip 140 can include a proximal end 142 and a distal end 144. Further, the safety clip 140 can include a ring handle 146 between the proximal end 142 of the safety clip 140 and the distal end 144 of the safety clip 140. In a particular embodiment, the safety clip 140 can be installed between the body 102 of the angioplasty balloon delivery device 100 and the handle 128 of the inner carrier catheter 120 such that the proximal end 142 of the safety clip 140 abuts the distal end 132 of the handle 128 and the distal end 144 of the safety clip 140 abuts the proximal end 104 of the body 102.

The safety clip 140 can fit over the inner carrier catheter 120. Further, the safety clip 140 can prevent the body 102 of the angioplasty balloon delivery device 100 from moving relative to the handle 128 of the inner carrier catheter 120. Further, the safety clip 140 can prevent the outer sheath 112 from sliding relative to the inner carrier catheter 120.

As shown in FIG. 1, the angioplasty balloon delivery device 100 can also include a valve 150 engaged with, or installed on, the proximal end 104 of the body 102 of the angioplasty balloon delivery device 100. In a particular embodiment, the valve 150 can be a Tuohy-Borst valve. When the valve 150 is tightened, e.g., by turning the valve 150 clockwise, the inner carrier catheter 120 cannot be moved relative to the outer sheath 112. However, when the valve 150 is loosened, e.g., by turning the valve 150 counterclockwise, the inner carrier catheter 120 can be moved relative to the outer sheath 112.

During use, the angioplasty balloon delivery device 100 can be threaded into a cardiovascular system of a patient to a target area. The radiopaque bands 128, 130 on the angioplasty balloon 126 can be used to guide the angioplasty balloon delivery device into the cardiovascular system of a patient, e.g., with the aid of fluoroscopy. The safety clip 140 can be removed from the inner carrier catheter 120 and the angioplasty balloon delivery device 100 and the valve 150 can be loosened. Thereafter, the body 102 of the angioplasty balloon delivery device 100 can be moved toward the handle of the inner carrier catheter 120 in order to slide the outer sheath 112 off of the angioplasty balloon 126 and expose the angioplasty balloon 126 inside the patient.

Once the angioplasty balloon 126 is exposed within the patient, the angioplasty balloon 126 can be inflated within the patient using a fluid, e.g., saline. After treatment is complete, the angioplasty balloon 126 can be deflated and retracted into the angioplasty balloon delivery device. Thereafter, the angioplasty balloon delivery device can be removed from the patient.

### Description of an Angioplasty Balloon

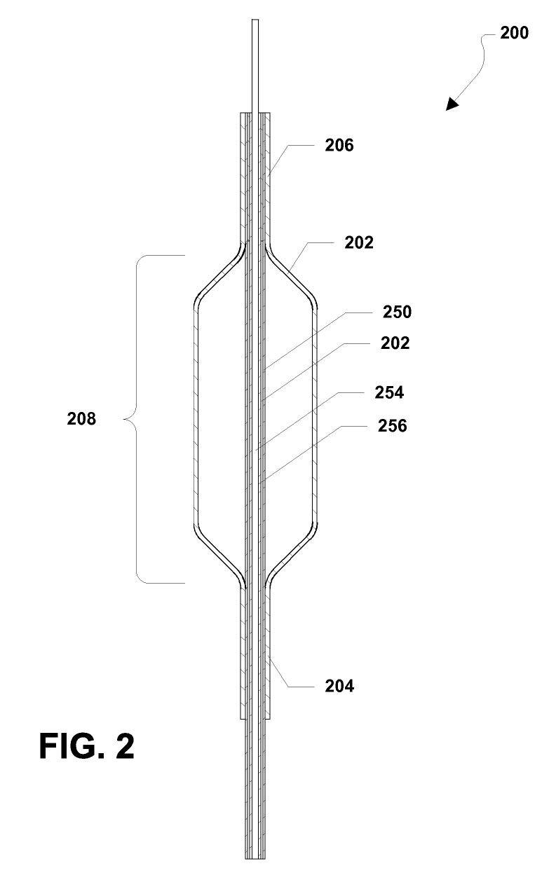

Referring to FIG. 2 through FIG. 4, a reinforced, non-compliant angioplasty balloon is shown and is generally designated 200. As shown, the angioplasty balloon 200 can include a body 202 having a first end 204 and a second end 206. Further, the body 202 can include an inflatable portion 208 that is established between the first end 204 and the second end 206. In a particular embodiment, the body 202 of the angioplasty balloon 200 can be made from a polymer material. For example, the polymer material can be polyurethane, polyethylene, nylon, PBAX, polyethylene terephthalate (PET), or a combination thereof.

In a particular embodiment, the inflatable portion 208 of the body 202 can have a thickness of at least one-tenths microns (0.1 μm). In another embodiment, the inflatable portion 208 of the body 202 can have a thickness of at least two-tenths microns (0.2 μm). In another embodiment, the inflatable portion 208 of the body 202 can have a thickness of at least three-tenths microns (0.3 μm). In another embodiment, the inflatable portion 208 of the body 202 can have a thickness of at least four-tenths microns (0.4 μm). In another embodiment, the inflatable portion 208 of the body 202 can have a thickness of at least five-tenths microns (0.5 μm).

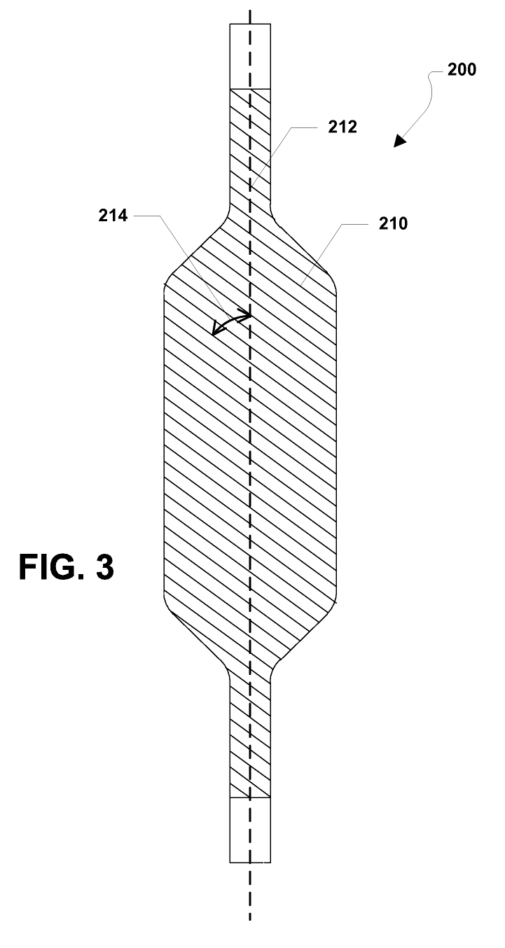

As shown in FIG. 3, the reinforced, non-compliant angioplasty balloon 200 can include a first reinforcing member 210 that can be wound around the body 202 of the angioplasty balloon 200. In a particular embodiment, the first reinforcing member can be a filament or a fiber made from ultra high weight polyethylene (UHWPE), Kevlar, aromatic polyamide (arimid), or a combination thereof. In a particular embodiment, the first reinforcing member 210 can be at least twenty deniers. In another embodiment, the first reinforcing member 210 can be at least twenty-five deniers. In yet another embodiment, the first reinforcing member 210 can be at least thirty deniers. In still another embodiment, the first reinforcing member 210 can be at least thirty-five deniers. In another embodiment, the first reinforcing member 210 can be at least forty deniers. In yet still another embodiment, the first reinforcing member 210 can be at least forty-five deniers. In another embodiment, the first reinforcing member 210 can be at least fifty deniers. In another embodiment, the first reinforcing member 210 is not greater than seventy-five deniers.

The angioplasty balloon 200 can define a longitudinal axis 212. Further, as indicated in FIG. 3, the first reinforcing member 210 can be wound around the body 202 of the angioplasty balloon an angle with respect to the longitudinal axis 212 to form a first winding angle 214. In a particular embodiment, the first winding angle 214 is in a range of fifteen degrees to forty-five degrees (15°-45°). In another embodiment, the first winding angle 214 is in a range of twenty degrees to forty degrees (20°-40°). In yet another embodiment, the first winding angle 214 is in a range of twenty-five degrees to thirty-five degrees (25°-35°). In another embodiment, the first winding angle 214 is approximately twenty-six and seven-tenths degrees (26.7°).

As indicated in FIG. 3, the first reinforcing member 210 can extend from the first end 204 of the body 202 to the second end 206 of the body 202 over the inflatable portion 208 of the body 202. In a particular embodiment, the first reinforcing member 210 can extend along one hundred percent (100%) of the body 202. In another embodiment, the first reinforcing member 210 can extend along at least ninety-five percent (95%) of the body 202. In still another embodiment, the first reinforcing member 210 can extend along at least ninety percent (90%) of the body 202. In yet another embodiment, the first reinforcing member 210 can extend along at least eighty-five percent (85%) of the body 202. In another embodiment, the first reinforcing member 210 can extend along at least eighty percent (80%) of the body 202. In still another embodiment, the first reinforcing member 210 does not extend over less than seventy-five percent (75%) of the body 202 of the angioplasty balloon 200.

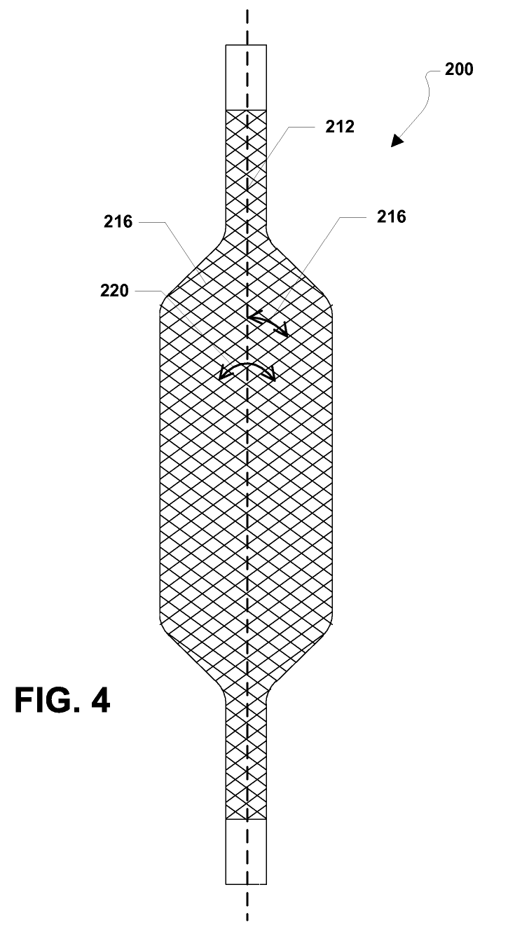

As illustrated in FIG. 4, a second reinforcing member 216 can also be wound around the body 202 of the angioplasty balloon 200. In a particular embodiment, the second reinforcing member 216 can be a filament or a fiber made from ultra high weight polyethylene (UHWPE), Kevlar, aromatic polyamide (arimid), or a combination thereof. In a particular embodiment, the second reinforcing member 216 can be at least twenty deniers. In another embodiment, the second reinforcing member 216 can be at least twenty-five deniers. In yet another embodiment, the second reinforcing member 216 can be at least thirty deniers. In still another embodiment, the second reinforcing member 216 can be at least thirty-five deniers. In another embodiment, the second reinforcing member 216 can be at least forty deniers. In yet still another embodiment, the second reinforcing member 216 can be at least forty-five deniers. In another embodiment, the second reinforcing member 216 can be at least fifty deniers. In another embodiment, the second reinforcing member 216 is not greater than seventy-five deniers.

The second reinforcing member 216 can also be wound at an angle with respect to the longitudinal axis 212 to form a second winding angle 218. In a particular embodiment, the second winding angle 218 is in a range of one hundred thirty-five degrees to one hundred sixty-five degrees (135°-165°). In another embodiment, the second winding angle 218 is in a range of one hundred forty degrees to one hundred sixty degrees (140°-160°). In yet another embodiment, the second winding angle 218 is in a range of one hundred forty-five degrees to one hundred fifty-five degrees (145°-155°). In another embodiment, the second winding angle 218 is approximately one hundred forty-six and seven-tenths degrees (146.7°).

FIG. 4 shows that the second reinforcing member 216 crosses the first reinforcing member 210 to form a crossing angle 220. In a particular embodiment, the crossing angle 220 is in a range of thirty degrees to ninety degrees (30°-90°). In another embodiment, the crossing angle 220 is in a range of thirty-five degrees to eighty-five degrees (35°-85°). In yet another embodiment, the crossing angle 220 is in a range of forty degrees to eighty degrees (40°-80°). In still another embodiment, the crossing angle 220 is in a range of forty-five degrees to seventy-five degrees (45°-75°). In another embodiment, the crossing angle 220 is in a range of fifty degrees to seventy degrees (50°-70°). In still yet another embodiment, the crossing angle 220 is approximately fifty-three and four tenths degrees (53.4°).

In a particular embodiment, the reinforced, non-compliant angioplasty balloon 200 can be bi-directional. In other words, the reinforced, non-compliant angioplasty balloon 200 can be installed on a catheter in either direction. Further, the first winding angle of the first reinforcing member 210 can be a mirrored opposite to the second winding angle of the second reinforcing member 216.

As indicated in FIG. 4, the second reinforcing member 216 can extend from the first end 204 of the body 202 to the second end 206 of the body 202 over the inflatable portion 208 of the body 202. In a particular embodiment, the second reinforcing member 216 can extend along one hundred percent (100%) of the body 202. In another embodiment, the second reinforcing member 216 can extend along at least ninety-five percent (95%) of the body 202. In still another embodiment, the second reinforcing member 216 can extend along at least ninety percent (90%) of the body 202. In yet another embodiment, the second reinforcing member 216 can extend along at least eighty-five percent (85%) of the body 202. In another embodiment, the second reinforcing member 216 can extend along at least eighty percent (80%) of the body 202. In still another embodiment, the second reinforcing member 216 does not extend over less than seventy-five percent (75%) of the body 202 of the angioplasty balloon 200.

The reinforcing members 210, 216 can have a tensile strength of at least 100×10³ pounds per square inch (psi). In another embodiment, the reinforcing members 210, 216 can have a tensile strength of at least 150×10³ psi. In yet another embodiment, the reinforcing members 210, 216 can have a tensile strength of at least 200×10³ psi. In another embodiment, the reinforcing members 210, 216 can have a tensile strength of at least 250×10³ psi. In still another embodiment, the reinforcing members 210, 216 can have a tensile strength of at least 300×10³ psi. In yet still another embodiment, the reinforcing members 210, 216 can have a tensile strength of at least 350×10³ psi. In another embodiment, the reinforcing members 210, 216 can have a tensile strength of at least 400×10³ psi. In another embodiment, the reinforcing members 210, 216 can have a tensile strength of at least 450×10³ psi. In another embodiment, the reinforcing members 210, 216 can have a tensile strength of at least 500×10³ psi. In yet another embodiment, the reinforcing members 210, 216 can have a tensile strength of at least 550×10³ psi. In another embodiment, the reinforcing members 210, 216 can have a tensile strength of at least 600×10³ psi. In another embodiment, the reinforcing members 210, 216 can have a tensile strength of at least 650×10³ psi. In another embodiment, the reinforcing members 210, 216 can have a tensile strength of at least 700×10³ psi. In another embodiment, the reinforcing members 210, 216 can have a tensile strength of at least 750×10³ psi. In still another embodiment, the tensile strength of the reinforcing members 210, 216 is not greater than 900×10³ psi.

In a particular embodiment, the reinforcing members 210, 216 can provide increased resistance to bursting when the reinforced, non-compliant angioplasty balloon 200 is inflated. For example, the reinforced, non-compliant angioplasty balloon 200 can be inflated to at least twenty atmospheres (20 atm). In another embodiment, the reinforced, non-compliant angioplasty balloon 200 can be inflated to at least thirty atmospheres (30 atm). In yet another embodiment, the reinforced, non-compliant angioplasty balloon 200 can be inflated to at least forty atmospheres (40 atm). In still another embodiment, the reinforced, non-compliant angioplasty balloon 200 can be inflated to at least fifty atmospheres (50 atm). In another embodiment, the reinforced, non-compliant angioplasty balloon 200 can be inflated to at least sixty atmospheres (60 atm). In another embodiment, the reinforced, non-compliant angioplasty balloon 200 can be inflated to at least seventy atmospheres (70 atm). In yet still another embodiment, the reinforced, non-compliant angioplasty balloon 200 can be inflated to at least eighty atmospheres (80 atm). In another embodiment, the reinforced, non-compliant angioplasty balloon 200 can be inflated to at least ninety atmospheres (90 atm). In another embodiment, the reinforced, non-compliant angioplasty balloon 200 is not inflated greater one hundred atmospheres (100 atm).

Referring back to FIG. 2, the reinforced, non-compliant angioplasty balloon 200 can be installed on a carrier catheter 250. For example, the ends 204, 206 of the body 202 of the reinforced, non-compliant angioplasty balloon 200 can be glued, or otherwise affixed, to the carrier catheter 250. The reinforced, non-compliant angioplasty balloon 200 can be installed so that either end 204, 206 is located near an end of the carrier catheter 250. As such, the reinforced, non-compliant angioplasty balloon 200 can be bidirectional.

FIG. 2 shows a guide wire catheter 252 installed within the carrier catheter 250. The guide wire catheter 252 can be sized and shaped to fit over a guide wire 254. Further, the guide wire catheter 252 can be sized so that a fluid communication path 256 is formed between the carrier catheter 250 and the guide wire catheter 252. The fluid communication path 256 can allow an inflating fluid to be delivered to the reinforced, non-compliant angioplasty balloon 200. The carrier catheter can be formed with a plurality of fluid ports to allow the fluid to be expressed from the fluid communication path 256 and into the interior of the reinforced, non-compliant angioplasty balloon 200, e.g., into the interior of the inflatable portion 208 of the reinforced, non-compliant angioplasty balloon 200.

### Description of a Method of Making an Angioplasty Balloon

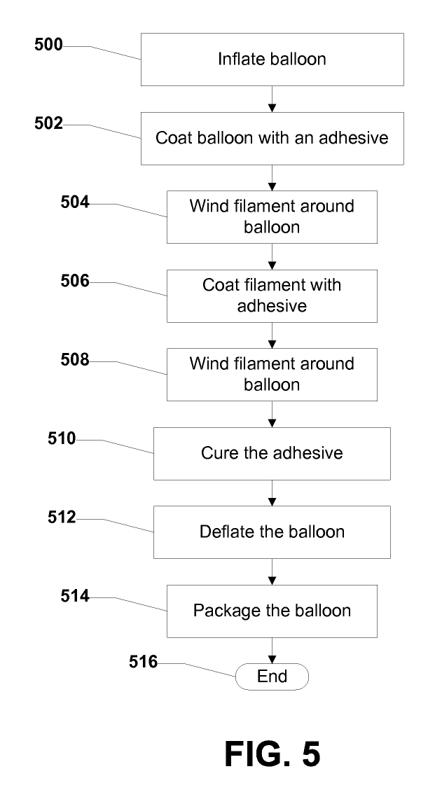

Referring now to FIG. 5, a method of making a reinforced, non-compliant angioplasty balloon is shown and commences at block 500. At block 500, an angioplasty balloon is inflated. At block 502, the angioplasty balloon can be coated with an adhesive. For example, the angioplasty balloon can be dip coated. Alternatively, the angioplasty balloon can be spray coated. In either case, the angioplasty balloon can be coated with a polymer adhesive. For example, the polymer adhesive can be PBAX.

Continuing to block 504, a filament can be wound around the angioplasty balloon. In a particular embodiment, the filament is wound around the angioplasty balloon at a first winding angle with respect to a longitudinal axis of the angioplasty balloon. The filament is wound from a first end of the angioplasty balloon to a second end of the angioplasty balloon.

In a particular embodiment, the first winding angle is in a range of fifteen degrees to forty-five degrees (15°-45°). In another embodiment, the first winding angle is in a range of twenty degrees to forty degrees (20°-40°). In yet another embodiment, the first winding angle is in a range of twenty-five degrees to thirty-five degrees (25°-35°). In another embodiment, the first winding angle is approximately twenty-six and seven-tenths degrees (26.7°).

Moving to block 506, the filament is, again, coated with an adhesive. For example, the angioplasty balloon can be dip coated. Alternatively, the angioplasty balloon can be spray coated. In either case, the angioplasty balloon can be coated with a polymer adhesive. For example, the polymer adhesive can be PBAX. Further, at block 508, the filament is wound around the balloon in a direction opposite to the first winding, e.g., from the second end of angioplasty balloon to the first end of the angioplasty balloon. The filament can be wound around the angioplasty balloon at a second winding angle with respect to the longitudinal axis of the angioplasty balloon.

In a particular embodiment, the second winding angle is in a range of one hundred thirty-five degrees to one hundred sixty-five degrees (135°-165°). In another embodiment, the second winding angle is in a range of one hundred forty degrees to one hundred sixty degrees (140°-160°). In yet another embodiment, the second winding angle is in a range of one hundred forty-five degrees to one hundred fifty-five degrees (145°-155°). In another embodiment, the second winding angle is approximately one hundred forty-six and seven-tenths degrees (146.7°).

In a particular embodiment, the second winding crosses the first winding to establish a crossing angle. In a particular embodiment, the crossing angle is in a range of thirty degrees to ninety degrees (30°-90°). In another embodiment, the crossing angle is in a range of thirty-five degrees to eighty-five degrees (35°-85°). In yet another embodiment, the crossing angle is in a range of forty degrees to eighty degrees (40°-80°). In still another embodiment, the crossing angle is in a range of forty-five degrees to seventy-five degrees (45°-75°). In another embodiment, the crossing angle is in a range of fifty degrees to seventy degrees (50°-70°). In still yet another embodiment, the crossing angle is approximately fifty-three and four tenths degrees (53.4°).

Continuing to block 510, the adhesive is cured. Thereafter, at block 512, the angioplasty balloon can be deflated. Further, at block 514, the angioplasty balloon can be packaged. Thereafter, the method can end at state 516. In another embodiment, in lieu of dip coating the angioplasty balloon, the filaments can be coated prior to being wound around the angioplasty balloon. In such an embodiment, the filaments can be dragged, or otherwise moved, through an adhesive-containing trough immediately prior to be wound around the angioplasty balloon.

In a particular embodiment, the reinforced, non-compliant angioplasty balloon can include a predetermined number of windings per filament per inch (in) length of the reinforced, non-compliant angioplasty balloon. For example, the reinforced, non-compliant angioplasty balloon can include at least ten windings per filament per inch (10/in). In another embodiment, the reinforced, non-compliant angioplasty balloon can include at least twenty windings per filament per inch (20/in). In yet another embodiment, reinforced, non-compliant angioplasty balloon can include at least thirty windings per filament per inch (30/in). In another embodiment, the reinforced, non-compliant angioplasty balloon can include at least forty windings per filament per inch (40/in). In another embodiment, the reinforced, non-compliant angioplasty balloon can include at least fifty windings per filament per inch (50/in). In another embodiment, the reinforced, non-compliant angioplasty balloon can include at least sixty windings per filament per inch (60/in). In another embodiment, the reinforced, non-compliant angioplasty balloon can include at least seventy windings per filament per inch (70/in). In another embodiment, the reinforced, non-compliant angioplasty balloon can include at least eighty windings per filament per inch (80/in). In another embodiment, the reinforced, non-compliant angioplasty balloon can include at least ninety windings per filament per inch (90/in). In another embodiment, the reinforced, non-compliant angioplasty balloon can include at least one hundred windings per filament per inch (100/in).

### Conclusion

Embodiments described herein provide an angioplasty balloon that can be used to treat vascular stenosis. Embodiments described herein can include a first reinforcing member and a second reinforcing member. The reinforcing members can increase a burst pressure of the angioplasty balloon. As such, embodiments herein can be used to treat stenosis using relatively high inflation pressures.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. An angioplasty balloon, comprising:
a body having a first end, a second end, and an inflatable portion established between the first end and the second end; and
a first reinforcing member wound around the body from the first end to the second end over the inflatable portion, wherein the first reinforcing member is wound at a first winding angle with respect to a longitudinal axis, and wherein the first winding angle is in a range of between about fifteen degrees to about forty-five degrees (15°-45°).

2. The angioplasty balloon of claim 1, wherein the first winding angle is in a range of between about twenty degrees to about forty degrees (20°-40°).

3. The angioplasty balloon of claim 2, wherein the first winding angle is in a range of twenty-five degrees to thirty-five degrees (25°-35°).

4. The angioplasty balloon of claim 3, wherein the first winding angle is approximately twenty-six degrees (26°).

5. The angioplasty balloon of claim 1, further comprising a second reinforcing member substantially wound around the body over the first reinforcing member from the first end to the second end over the inflatable portion.

6. The angioplasty balloon of claim 5, wherein the second reinforcing member is wound at a second winding angle with respect to the longitudinal axis and wherein the second winding angle is in a range of between about fifteen degrees to about forty-five degrees (15°-45°).

7. The angioplasty balloon of claim 6, wherein the second winding angle is in a range of twenty degrees to forty degrees (20°-40°).

8. The angioplasty balloon of claim 7, wherein the second winding angle is in a range of twenty-five degrees to thirty-five degrees (25°-35°).

9. The angioplasty balloon of claim 8, wherein the second winding angle is approximately twenty-six degrees (26°).

10. (canceled)

11. (canceled)

12. (canceled)

13. (canceled)

14. (canceled)

15. (canceled)

16. (canceled)

17. An angioplasty balloon, comprising:
a body having a first end, a second end, and an inflatable portion established between the first end and the second end;
a first reinforcing member substantially wound around the body from the first end to the second end over the inflatable portion; and
a second reinforcing member substantially wound around the body from the second end to the first end over the first reinforcing member, wherein the second reinforcing member crosses the first reinforcing member at a crossing angle and wherein the crossing angle is in a range of between about thirty degrees to about ninety degrees (30°-90°).

18. The angioplasty balloon of claim 17, wherein the crossing angle is in a range of thirty-five degrees to eighty-five degrees (35°-85°).

19. The angioplasty balloon of claim 18, wherein the crossing angle is in a range of forty degrees to eighty degrees (40°-80°).

20. The angioplasty balloon of claim 19, wherein the crossing angle is in a range of forty-five degrees to seventy-five degrees (45°-75°).

21. The angioplasty balloon of claim 20, wherein the crossing angle is in a range of fifty degrees to seventy degrees (50°-70°).

22. (canceled)

23. (canceled)

24. (canceled)

25. (canceled)

26. (canceled)

27. (canceled)

28. (canceled)

29. (canceled)

30. (canceled)

31. The angioplasty balloon of claim 17, wherein the angioplasty balloon is inflatable to a pressure of at least about fifty atmospheres (50 atm).

32. The angioplasty balloon of claim 3 1, wherein the angioplasty balloon is inflatable to a pressure of at least about sixty atmospheres (60 atm).

33. The angioplasty balloon of claim 32, wherein the angioplasty balloon is inflatable to a pressure of at least about seventy atmospheres (70 atm).

34. The angioplasty balloon of claim 33, wherein the angioplasty balloon is inflatable to a pressure of at least about eighty atmospheres (80 atm).

35. The angioplasty balloon of claim 33, wherein the angioplasty balloon is inflatable to a pressure not greater than one hundred atmospheres (100 atm).

36. (canceled)

37. A method of making an angioplasty balloon, comprising:
inflating an angioplasty balloon;
coating the angioplasty balloon, a first reinforcing member, or a combination thereof with an adhesive; and
winding the first reinforcing member around the angioplasty balloon at a first winding angle with respect to a longitudinal axis of the angioplasty balloon, wherein the first winding angle is in a range of between about fifteen degrees to about forty-five degrees (15°-45°).

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)
